(19) United States
(12) Patent Application Publication
(10) Pub. No.: US 2010/0159498 A1
(43) Pub. Date: Jun. 24, 2010

(54) BLOOD ANALYZER WITH A BLOOD CELL SEDIMENTATION CONTROL MECHANISM AND METHOD OF USE

(76) Inventors: Kalle RITZEN, Stockholm (SE); Magdalena Nilsson, Vendelso (SE); Sherb Edmondson, Sunnyvale, CA (US); Wolfgang Magnusson, Huddinge (SE); Gunnar Magnusson, Arsta (SE)

Correspondence Address: LATIMER & MAYBERRY IP LAW, LLP, 13873 PARK CENTER ROAD, SUITE 106, HERNDON, VA, 20171, US

(21) Appl. No.: 12/339,398

(22) Filed: Dec. 19, 2008

(51) Int. Cl. C12Q 1/02; C12M 1/34

(52) U.S. Cl. 435/29; 435/286.1; 435/286.5

(57) ABSTRACT

A blood analyzer having a blood cell sedimentation control mechanism is disclosed, which includes a cassette receiving interface including a cassette compartment and a blood sensor operable to detect a presence of blood in a disposable cassette removably disposed within the cassette compartment; a system control electrically connected to the blood sensor, and a blood measurement assembly connected to the system control and adapted to connect with the disposable cassette. The system control includes a time recording mechanism and a predetermined sedimentation time control criterion. Further disclosed is a method of controlling blood cell sedimentation during sample preparation process on the blood analyzer.

### BACKGROUND OF THE INVENTION

1. Field of the Invention

The present invention relates to a blood analyzer having a blood cell sedimentation control mechanism and a method of controlling blood cell sedimentation during sample preparation process on a blood analyzer.

2. Background of the Invention

Red blood cell and white blood cell concentrations of a blood sample, also commonly referred to red blood cell count (RBC) and white blood cell count (WBC), are important clinical diagnosis parameters. On hematology analyzers, the red blood cell concentration is typically measured with impedance or light scatter measurements using an aliquot of a whole blood sample substantially diluted with a blood diluent, and the white blood cell concentration is typically measured with impedance or light scatter measurements using another aliquot of the whole blood sample mixed with a lysing reagent to lyse red blood cells, yet maintaining the white blood cells to a certain degree for measurement.

On fully automated hematology analyzers, the whole blood samples are continuously mixed prior to aspirating the blood into the instrument. After aspiration, two or more predetermined volumes of the blood are segmented, each thereof is immediately mixed with a reagent for a specific measurement, for example, measurements of red blood cell concentration, white blood cell concentration, and hemoglobin concentration, respectively. During the automated actions, the blood does not have idle or standing time; therefore, the effect of sedimentation to the accuracy of the measurement is not a practical concern.

However, on semi-automated hematology analyzers, where sample preparation process involves manual operation by a technician, a blood sample may be idle, or standing, for a period of time in one or more process steps, during which sedimentation of the blood cells may occur. Typically, the length of the idle time is not monitored or controlled, and is operator dependent.

During the idle or standing time, the red blood cells and white blood cells descend, driven by gravity. Other particles, such as platelets, may move upward instead. Consequently, at different parts of the blood in the vertical direction, the concentrations of the blood cells can be different. As such, in a subsequent step of segmenting a portion of the blood for measurement, the cell concentration in the segmented portion may not represent the original concentration of that cell type in the whole blood. As the degree of sedimentation increases, it may lead to erroneous measurement results.

Therefore, it is desirable to have a hematology analyzer that has a mechanism for controlling the effect of sedimentation during sample preparation, and hence, to reduce operator dependency and ensure accuracy of the measurement on the blood analyzer.

### SUMMARY OF THE INVENTION

In one aspect, the present invention is directed to a blood analyzer having a blood cell sedimentation control mechanism. In one embodiment, the blood analyzer comprises a cassette receiving interface comprising a cassette compartment and a blood sensor operable to detect a presence of blood in a disposable cassette removably disposed within the cassette compartment; a system control connected to the blood sensor, and a blood measurement assembly connected to the system control, and adapted to connect with the disposable cassette. The system control comprises a time recording mechanism and a predetermined sedimentation time control criterion. The sedimentation time control criterion comprises an upper limit of a dwelling time defined as a time period between a filling time at which the blood sensor detects a blood sample being filled into the cassette and a sampling time at which a predetermined volume of the blood sample is isolated in the cassette for measurement.

The system control further comprises a sedimentation evaluation mechanism operable to evaluate a recorded dwelling time of the blood sample in reference to the predetermined sedimentation control criterion, and predetermined sample analysis instructions, including a proceed-further instruction, a flagging instruction, or an abortion instruction.

In one embodiment, the cassette receiving interface is movable between a first position and a second position, and the blood analyzer further comprises a position sensor electrically connected to the system control, operable to detect the position of the cassette receiving interface. The cassette receiving interface further comprises a cassette sensor, electrically connected to the system control, operable to detect the presence of the disposable cassette in the cassette compartment.

In a further embodiment, the sedimentation time control criterion comprises an upper limit of a first dwelling time and an upper limit of a second dwelling time. The first dwelling time is defined as a time period between a filling time at which the blood sensor detects a blood sample being filled into the cassette and an engaging time at which the cassette receiving interface is moved to the second position. The second dwelling time is defined as a time period between the engaging time and a sampling time at which a predetermined volume of the blood sample is isolated in the cassette for measurement.

In a further aspect, the present invention provides a method of controlling blood cell sedimentation during sample preparation process on a blood analyzer. In one embodiment, the method comprises providing a blood analyzer comprising a cassette receiving interface that includes a cassette compartment and a blood sensor, a blood measurement assembly, and a system control electrically connected to the blood sensor and the blood measurement assembly, the system control comprising a time recording mechanism and a predetermined sedimentation time control criterion; placing a disposable cassette into the cassette compartment, and filling a blood sample into the disposable cassette; isolating a predetermined volume of the blood sample in the cassette; recording a dwelling time using the time recording mechanism; comparing recorded dwelling time of the blood sample with an upper limit of the dwelling time in the predetermined sedimentation time control criterion; and generating a sample analysis decision based on a result of the comparison or evaluation.

The advantages of the present invention will become apparent from the following description taken in conjunction with the accompanying drawings showing exemplary embodiments of the invention.

### BRIEF DESCRIPTION OF THE DRAWINGS

FIGS. 1A and 1BA are illustrative perspective views of the blood analyzer in one embodiment of the present invention, with the cassette receiving interface in closed and open positions, respectively.

FIG. 2 is a front perspective view of the cassette receiving interface of the blood analyzer shown in FIG. 1A in a horizontal position.

FIG. 3 is a front perspective view of the cassette receiving interface of the blood analyzer shown in FIG. 1A in a horizontal position and having a disposable cassette placed within the cassette compartment of the cassette receiving interface.

FIG. 4 is a perspective view of the blood analyzer of a further embodiment of the present invention, wherein the cassette receiving interface is in a form of a movable tray, at its open position.

FIG. 5 is an illustrative cross-sectional view showing the detection area in the sampling section of a disposable cassette, the light source and the light detector of the blood sensor in one embodiment of the present invention.

FIG. 6 is a perspective view of the disposable cassette shown in FIG. 3.

FIG. 7 is a top view of the disposable cassette shown in FIG. 3.

FIG. 8 is a perspective of the sampling sled of the disposable cassette shown in FIG. 6.

FIG. 9 is a bottom perspective view of the sampling gasket of the disposable cassette.

FIG. 10 is an enlarged cross-sectional view of the sampling section of the disposable cassette, along line 2-2′ of FIG. 11, showing communications among the filling inlet, the first and second sampling cavities and the venting aperture at the filling position.

FIGS. 11A and 11BA are illustrative see-through views of the sampling section of the disposable cassette, with the sampling sled at the filling position and the flushing position, respectively.

FIG. 12 is an illustrative view showing the engagement of the disposable cassette with the piercing elements of the cassette interface of the blood measurement assembly of the blood analyzer.

FIG. 13A shows an embodiment in which the cassette has a pair of electrodes disposed within vent opening on the upper paned as an electrical sensing mechanism for the electrical sensor type of the blood sensor.

FIG. 13B shows the sampling section of the disposable cassette after a blood sample is filled in.

FIGS. 14A and 14B are enlarged partial cross sectional views of the sampling section of the disposable cassette, along line A-A′ of the sampling sled in FIG. 8, with the cassette at horizontal and vertical positions, respectively, showing a blood sample filled in the first sampling cavity of the sampling sled and in the recess of the sampling gasket.

FIG. 15 shows the obtained red blood cell concentration (RBC) with different dwelling times, and dependency of the sedimentation effect on the concentration of the red blood cells in the blood samples.

It is noted that in the drawings like numerals refer to like components.

### DETAILED DESCRIPTION OF EXEMPLARY EMBODIMENTS OF THE INVENTION

In one aspect, the present invention provides a blood analyzer having a blood cell sedimentation control mechanism.

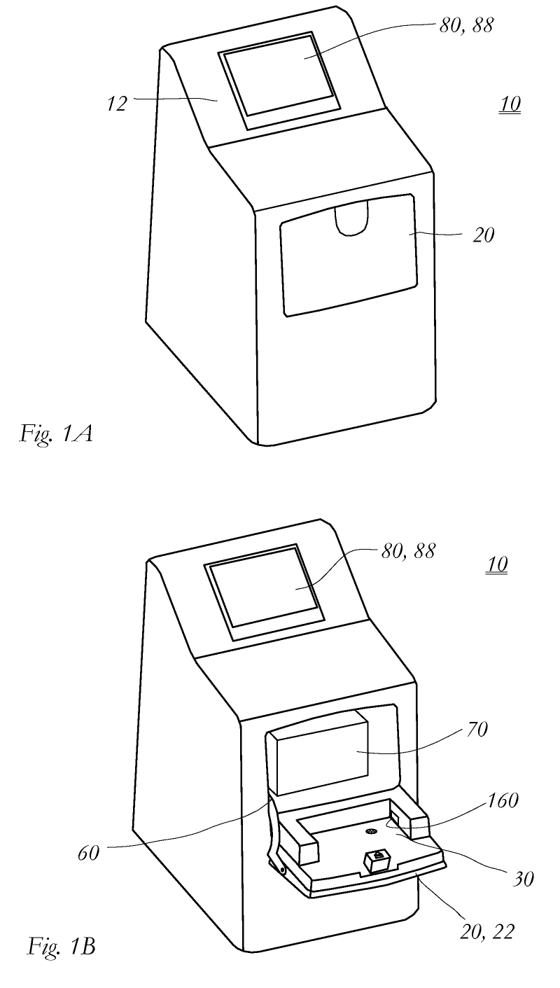

Referring to FIGS. 1 through 3, in one embodiment, the blood analyzer 10 of the present invention comprises a system housing 12, a cassette receiving interface 20, a blood measurement assembly 70, a system control 80, and a user interface 88.

In the embodiment shown FIGS. 1A and 1B, the cassette receiving interface 20 is in the form of a door, and is movable between an open position and a closed position, also referred to as first and second positions. Cassette receiving interface 20 comprises a door panel 22, a cassette compartment 30, and a blood sensor 40 operable to detect the presence of blood in a disposable cassette that is removably disposed within cassette compartment 30 during the measurement of a blood sample.

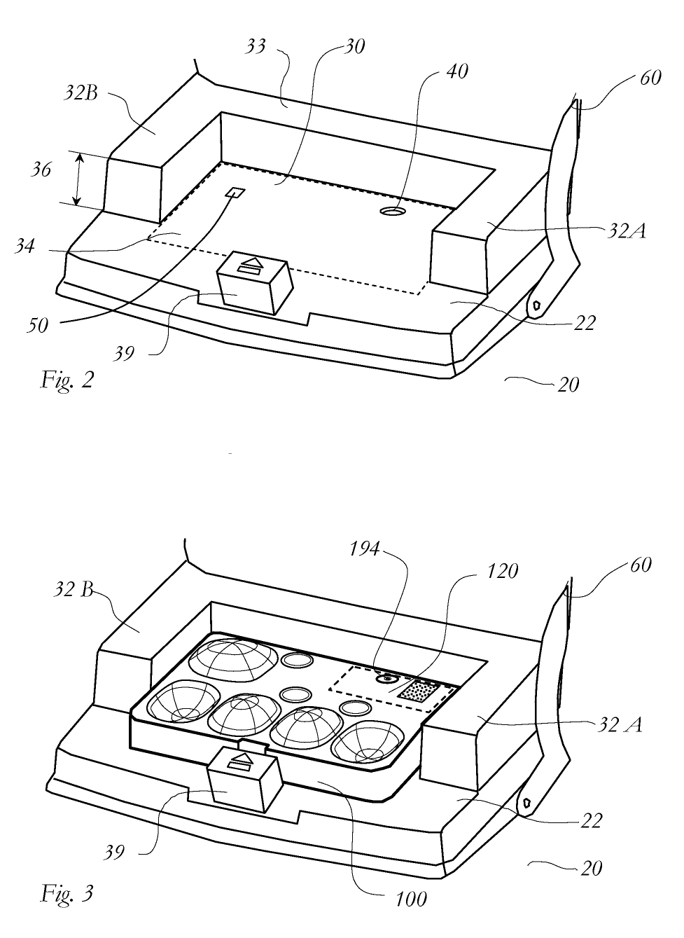

FIG. 2 shows cassette receiving interface 20 in an open, horizontal position, and FIG. 3 shows cassette receiving interface 20 in the same position with a disposable cassette 100 placed within cassette compartment 30. As shown in FIG. 2, cassette compartment 30 is formed by two side walls 32A and 32B, a rear wall 33 and a front stopper 39 on a substantially planar base 34. In the embodiment shown, base 34 is the interior surface of door panel 22; however, the cassette compartment can also be a separate unit from the door panel. Cassette compartment 30 has a width between the two side walls complimentary to the width of disposable cassette 100. Preferably, the height 36 of the walls in dimension is larger than the thickness of the cassette. With the structure and dimensions of cassette compartment 30, the disposable cassette is firmly held within the compartment during sample preparation process carried out by the blood analyzer.

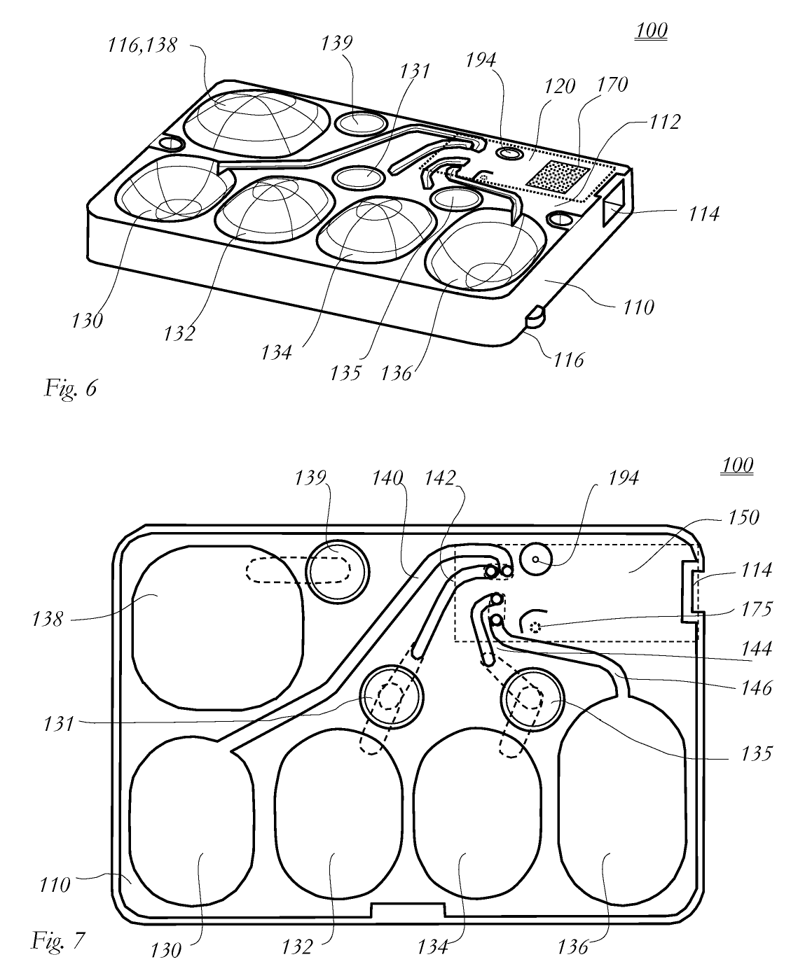

In the embodiment shown in FIG. 2, blood sensor 40 is an optical sensor, which includes a light detector 44, and preferably also includes a light source 42, as shown in FIG. 5. In the embodiment shown in FIG. 2, both light source 42 and light detector 44 are located in door panel 22, underneath base 34 of cassette compartment 30. FIG. 5 illustrates a partial cross-section of sampling section 120 of a disposable cassette 100 relative to light source 42 and light detector 44 in one embodiment of the present invention. The structure of cassette 100 is shown in FIGS. 6 and 7, and will be described in further detail later. Preferably, the housing of the disposable cassette and a sampling sled 150 disposed in the sampling section 120 are made of transparent materials.

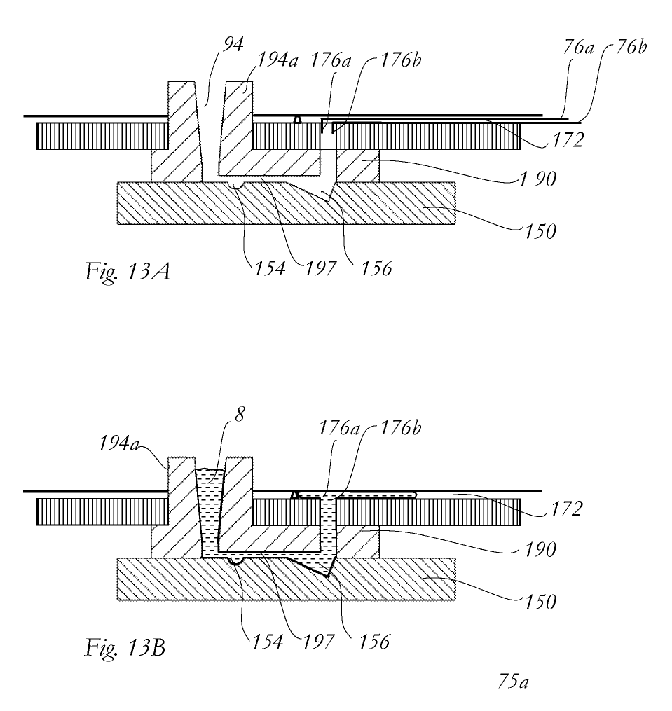

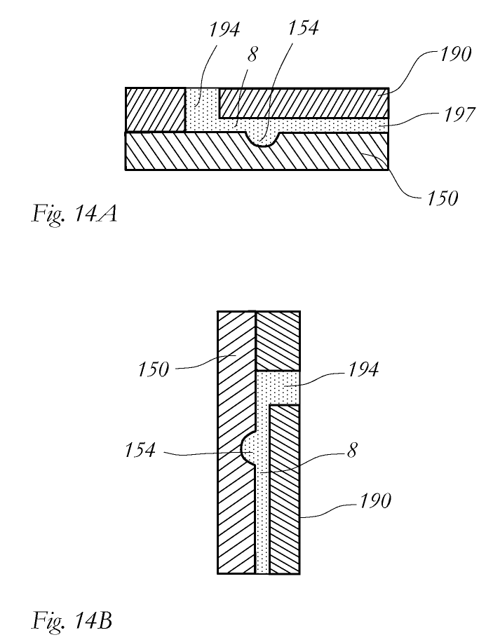

As shown in FIG. 5, light source 42, from underneath sampling sled 150 of disposable cassette 100, projects a light onto a detection area 46 in sampling section of the cassette, the light at this area is detected by light detector 44. The detection area 46 is selected from an area in sampling section 120, where the surface is covered by blood when a blood sample is filled in and the area is without light obstruction. The detection area can be from about 1 mm² to about 100 mm². When the blood analyzer is in operation, a blood sample is filled in through filling inlet 194 of a sampling gasket 190 into the space between sampling sled 150 and sampling gasket 190 of the cassette (also see FIG. 13A). The blood covers the surface of detection area 46, which absorbs light and causes a reduction of the light sensed by light detector 44. The light intensity change indicates the presence of a blood. As can be appreciated, since the housing and sampling sled are transparent, detection area 46 receives a certain level of light from the environment; however, the light intensity from the natural light source varies with the environment. Using the light source 42, which has a substantially stronger intensity than the light from the environment, the detection is more consistent and free of influence from the environment.

Light source 42 and light detector 44 can have various different arrangements, so long as the blood sensor enables a sensitive detection of the presence of a blood in the blood sampling section of the cassette. In the embodiment shown in FIG. 5, the angle α between the axis of the incident light and the vertical axis (which is 90° from the surface of base 34) and the angle β between the axis of the detected light and the vertical axis are both about 45°. In general, angle α can be in a range from about 0° to 90°, angle β can be in a range from about 0° to less than 80°, and these two angles do not need to be the same. For example, in one configuration, angle α is about 0° and angle β is about 45°. In this configuration, light source 42 projects light straight upward. In another configuration, angle α is about 45° and angle β is at 0°. In this configuration, light source 42 projects light from side and light detector 44 detects the light directly underneath detection area 46. Moreover, in an alternative arrangement, the incident light can be emitted horizontally in reference to base 34 of cassette compartment 30, and then reflected by a mirror to project onto detection area 46.

Various light sources and light detectors known in the art can be used for the purpose of the present invention. Suitable examples of the light source include, but not limited to, LED, laser, and lamp, and suitable examples of the light detector include, but not limited to, photodiode, phototransistor, photosensor array, and CCD array.

Light detector 44 of blood sensor 40 is connected to system control 80 and a time recording mechanism thereof, and the signal produced by light detector 44 can be used for determining a blood dwelling time, which will be described hereinafter in detail.

In another embodiment, the blood sensor is an electrical sensor disposed at a suitable location of cassette receiving interface 20, such as on the side wall or on the rear wall of cassette compartment 30. The electrical sensor is adapted to connect to a sensing mechanism in the disposable cassette that is to be placed in cassette receiving interface 20 for measurement of a blood sample on the blood analyzer. The sensing mechanism in one embodiment of the disposable cassette is described hereinafter in reference to FIG. 13A.

Preferably, cassette receiving interface 20 further comprises a cassette sensor 50 (see FIG. 2), operable to detect the presence or absence of a disposable cassette within cassette compartment 30. Cassette sensor 50 can be a mechanical, electrical or optical sensor, positioned at a suitable location of cassette compartment 30, for example, on base 34, side walls 32A or 32B, or rear wall 33. In the embodiment shown in FIG. 2, cassette sensor 50 is a mechanical sensor positioned on based 34. Cassette sensor 50 is electrically connected to system control 80, and the signal indicating a presence or absence of a disposable cassette within cassette compartment 30 can be used by system control 80 in controlling operation of the blood analyzer, which will be described in further detail hereinafter.

Blood analyzer 10 further comprises a position sensor 60, operable to detect the position of cassette receiving interface 20. Position sensor 60 can be a mechanical, electrical, or optical sensor, positioned at a suitable location of cassette receiving interface 20, such as around periphery thereof, or at a suitable location around the front opening 14 of system housing 12. In the embodiment shown in FIG. 2, position sensor 60 is located at the end of door hinge. Position sensor 60 detects cassette receiving interface 20 in the closed or open position. Position sensor 60 is electrically connected to system control 80, and the signal indicating an open or closed position of cassette receiving interface 20 can be used by system control 80 in controlling operation of the blood analyzer, which will be described in further detail hereinafter.

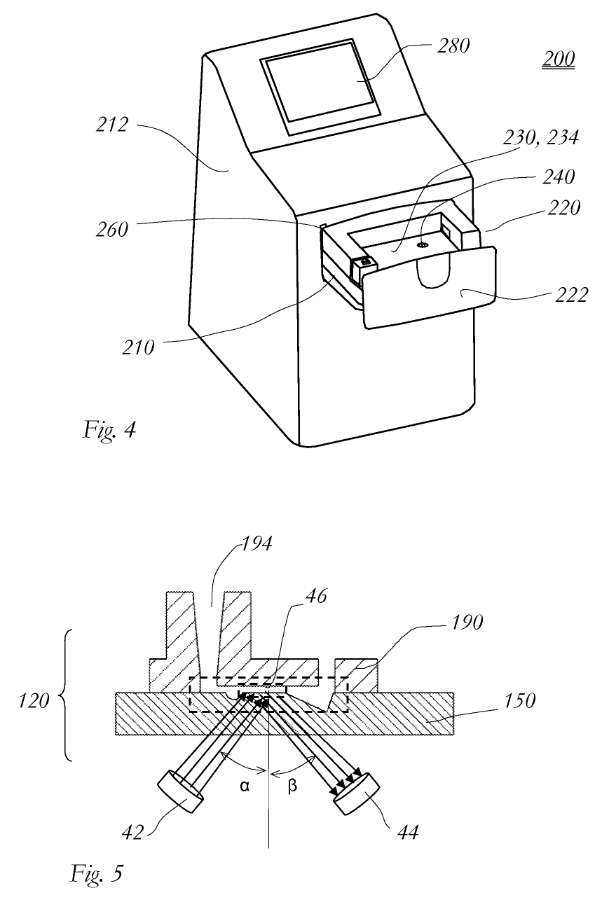

FIG. 4 shows a blood analyzer 200 in a further embodiment of the present invention. As shown, blood analyzer 200 includes a cassette receiving interface 220 in a form of sliding tray. Cassette receiving interface 220 has a front panel 222, support 210 having a sliding mechanism underneath (not shown) similar to that used for opening and closing a compact disk driver. There is a cassette compartment 230 disposed above support panel 210. The structure of cassette compartment 230 is similar to cassette compartment 30 of blood analyzer 10, with a base 234 and sidewalls, and dimensions of cassette compartment 230 is substantially the same as those of cassette compartment 30. When cassette receiving interface 220 is in its open position as shown in FIG. 4, disposable cassette 100 can be placed inside cassette compartment 230. When cassette receiving interface 220 is closed by sliding into system housing 212 of the blood analyzer 200, cassette compartment 230 is rotated to a vertical position by a rotation mechanism (not shown), which brings cassette 100 to the same orientation as it is in blood analyzer 10 when cassette receiving interface 20 is at its closed position.

In this embodiment, blood sensor 240 can have the same structure of blood sensor 40 of blood analyzer 10. Position sensor 260 is positioned on the upper edge of the front opening of system housing 212, which can be a mechanical, electrical or optical sensor. When cassette receiving interface 220 is closed, a direct contact of front panel 222 to position sensor 260, or light obstruction by front panel 222, triggers the sensor to indicate that cassette receiving interface 220 is closed. Then, system control 280, electrically connected to the sensor, activates the rotation mechanism to rotate cassette compartment 230 to the vertical position. Therefore, in this embodiment, for the purpose of monitoring sedimentation the first position of the cassette receiving interface is at its open position as shown in FIG. 4 and the second position is when cassette compartment 230 is in the vertical position. Other than the cassette receiving interface, in this embodiment the blood measurement assembly, pressure actuator assembly, system control, and user interface are substantially the same as those of blood analyzer 10, which are described in further detail hereinafter.

Blood measurement assembly 70 comprises one or more blood measurement devices operable to measure blood cells and/or contents thereof in a blood sample. In one embodiment, blood measurement assembly 70 comprises two blood measurement devices, one of which is used for measuring red blood cells and platelets of a blood sample and the other is used for measuring white blood cells of the blood sample. The blood measurement device comprises a flow path having an aperture, and a detector disposed adjacent to the aperture to detect individual cells passing through the aperture. The detector can be either an electrical detector or an optical detector. The electrical detector measures direct current impedance signals (DC), or radio frequency impedance signals (RF), generated when each blood cell suspended in an aqueous conductive sample mixture passes through the aperture. The impedance signals are used for counting number of cells and determining size of the cells in the sample mixture. The optical detector measures light scatter or absorption signals generated by blood cells passing through the aperture and these signals are used for counting number of cells and determining size of the cells in the sample mixture. Suitable electrical detectors and optical detectors known in the art for measuring blood cells can be used for the purpose of the present invention.

Blood measurement assembly 70 further comprises a hemoglobin measurement device, which comprises a cuvette with a light path of a determined length, a light source, and an optical detector in alignment with the light path to measure absorption of light passing through the cuvette. Preferably, the cuvette is fluidly connected with the blood measurement device that is used for measuring white blood cells, as such hemoglobin concentration and the white blood cells of a blood sample can be measured using one sample mixture. In measuring white blood cells and hemoglobin concentration, a volume of a blood sample is mixed with a lysing reagent to lyse red blood cells and release hemoglobin molecules, which form a hemoglobin chromogen, typically with a hemoglobin ligand or stabilizer contained in the lysing reagent. The formed sample mixture is passed through the aperture of the flow path, as well as the cuvette, and the white blood cells and hemoglobin concentration can be measured sequentially using the same sample mixture.

Alternatively, two separate sample mixtures can be prepared and used for measuring the white blood cells and hemoglobin concentration. In this arrangement, the hemoglobin measurement device is separated from the flow path that is used for measuring white blood cells.

The signals generated in measuring red blood cells, white blood cells and hemoglobin concentration are processed by a data processor, which can be either independent, or integrated into system control 80.

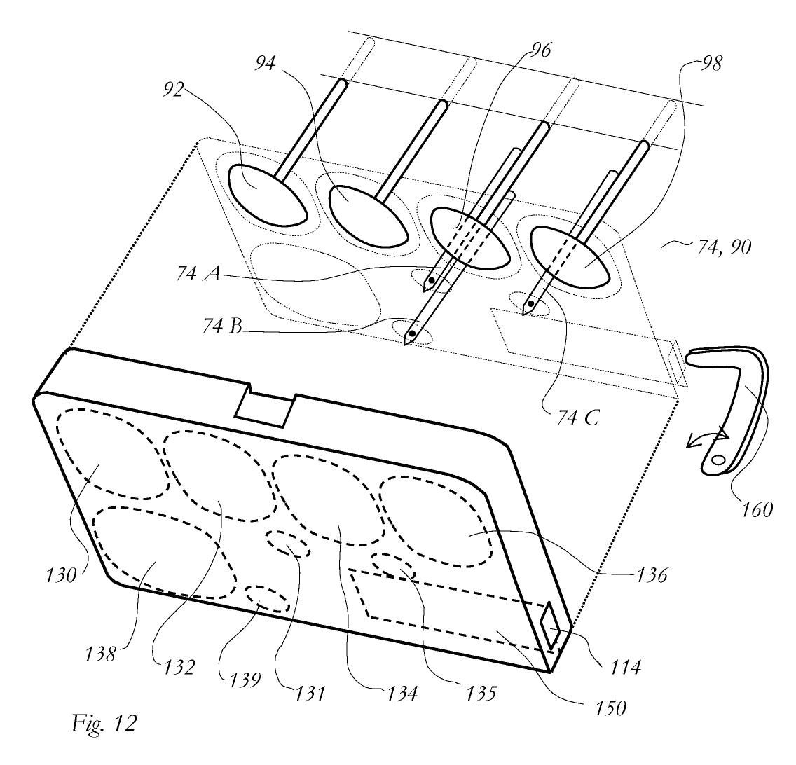

Blood measurement assembly 70 further comprises a cassette interface that is adapted to fluidly connect with disposable cassette 100, and cause delivery of a prepared sample mixture in disposable cassette 100 to blood measurement assembly 70 for measurement. In one embodiment as shown in FIG. 12, cassette interface 74 comprises one or more piercing elements, such as needles 74A, 74B, and 74C, operable to engage with sample outlets and cleaner outlet of disposable cassette 100 by piercing, which is further described hereinafter.

In one embodiment, blood analyzer 10 or 200 further comprises a pressure actuator assembly 90 adapted to apply a pressure on selected chambers to mix a blood with a reagent to prepare a sample mixture for measurement, as described further hereinafter. In one embodiment as shown in FIG. 12, pressure actuator assembly 90 includes multiple plungers 92, 94, 96, and 98, which are controlled by one or more motors (not shown). Each plunger has a mushroom head adapted to press against one of the chambers of disposable cassette 100.

For the purpose of understanding the blood cell sedimentation control mechanism of the blood analyzers of the present invention, an example disposable cassette that can be used on blood analyzer 10 or 200 is described hereinafter.

As shown in FIG. 6, disposable cassette 100 comprises a housing 110 having an upper panel 112 and a sampling section 120 having a filling inlet 194; multiple chambers or receptacles 130, 132, 134, 136, and 138, each formed by a depression of upper panel 112 of housing 110 and sealed by a diaphragm 116; and plurality of channels 140, 142, 144, and 146 adapted to interconnect selected chambers. In one embodiment, chambers 130 and 132 are interconnected as a pair for preparing a red blood cell sample mixture, wherein one of the two chambers, such as chamber 132 shown in FIG. 6, is pre-filled with a predetermined amount of a blood diluent. Similarly, chambers 134 and 136 are interconnected as a pair for preparing a white blood cell sample mixture, wherein one of the two chambers, such as chamber 134 shown in FIG. 6, is pre-filled with a predetermined amount of a lytic reagent. In the embodiment shown, chamber 138 is pre-filled with a cleaning solution for cleaning the flow paths of the blood measurement devices of blood measurement assembly 70 after measurement of a blood sample. Preferably, diaphragm 116 seals the entire upper side of upper panel 112, which is welded onto elevated boarders around the chambers and around the channels; however, there is a space 172 between the diaphragm and the upper side of upper panel 112 at sampling section 120, particularly above a vent opening 175 of upper panel 112 for releasing air in the sampling section during blood filling (see FIGS. 7 and 10).

Disposable cassette 100 further includes sample outlets 131 and 135, the former is interconnected with chambers 132 and channel 142 and the latter is interconnected with chambers 134 and channel 144. Each sample outlet includes a divider within, which seals the liquid reagent contained in chambers 132 and 134 from flowing out. The cassette also has a cleaner outlet 139 connected to chamber 138. Optionally, disposable cassette 100 can also have a bar code 170 for identifying each cassette.

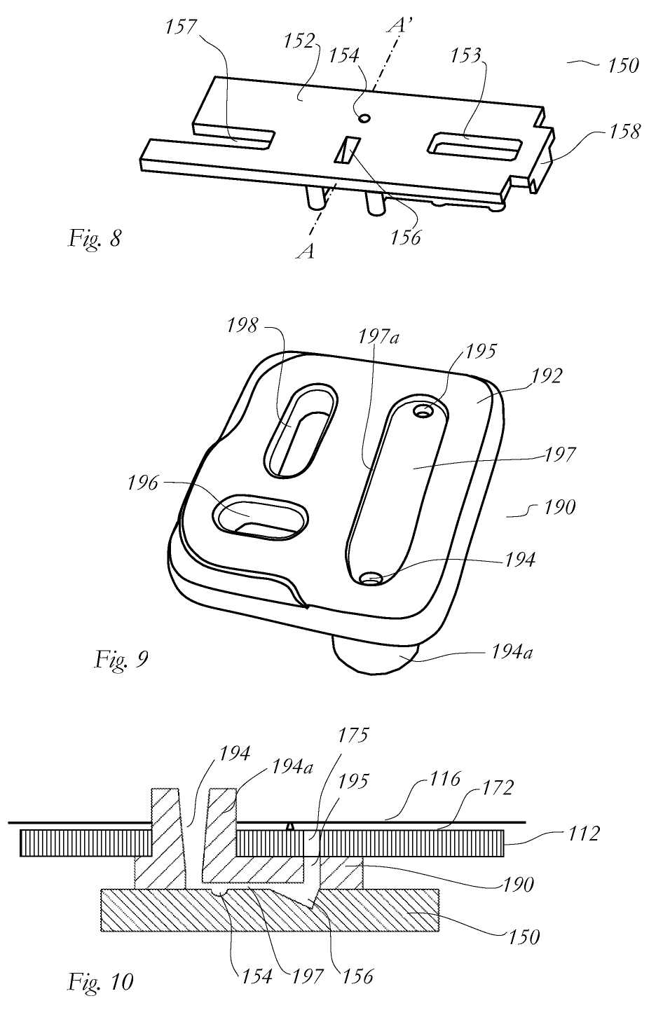

In one embodiment, disposable cassette 100 comprises a sampling sled 150 in sampling section 120, movable between a filling position and a flushing position (see FIGS. 7 and 8). As shown in FIG. 8, sampling sled 150 has a flat upper surface 152, a first sampling cavity 154, and a second sampling cavity 156. Both sampling cavities are in a form of recess on the upper surface, and each has a predetermined volume. Sampling cavity 154 is used to isolate a predetermined volume of a blood sample for red blood cell measurement and sampling cavity 156 is used to isolate a predetermined volume of a blood sample for white blood cell measurement. In one exemplary embodiment, sampling cavity 154 has a volume about 0.1 microliter and sampling cavity 156 has a volume about 5 microliter. Because concentration of the red blood cells in a blood sample is substantially higher than concentration of the white blood cells, sampling cavity 154 is substantially smaller than sampling cavity 156. Sampling sled 150 is snap fit onto the lower side of upper panel 112 of the housing through slots 157 and 155. Sampling sled 150 has a pusher interface 158, which can be accessed through a pusher opening 114 of housing 20 (see FIG. 6).

Disposable cassette 100 further includes a sampling gasket 190 as shown in FIG. 9, which is disposed within a gasket seat on the lower side of upper panel 112. Sampling gasket 190 has a flat lower surface 192, which is directly against flat upper surface 152 of sampling sled 150. On lower surface 192, there is an elongated recess 197 extending from the outer side of filling inlet 194 to the outer side of venting aperture 195. Since the flat lower surface 192 is against the flat upper surface 152 of sampling sled 150, recess 197 forms a blood filling space. Sampling gasket 190 includes a filling inlet 194 surrounded by a circular rim 194a and a venting aperture 195. Filling inlet 194 is directly accessible from the upper side of cassette 100 for filling a blood sample. Sampling gasket 190 includes a first through-hole 196, which connects to channel 140 and 142 and a second through-hole 198, which connects to channel 144 and 146.

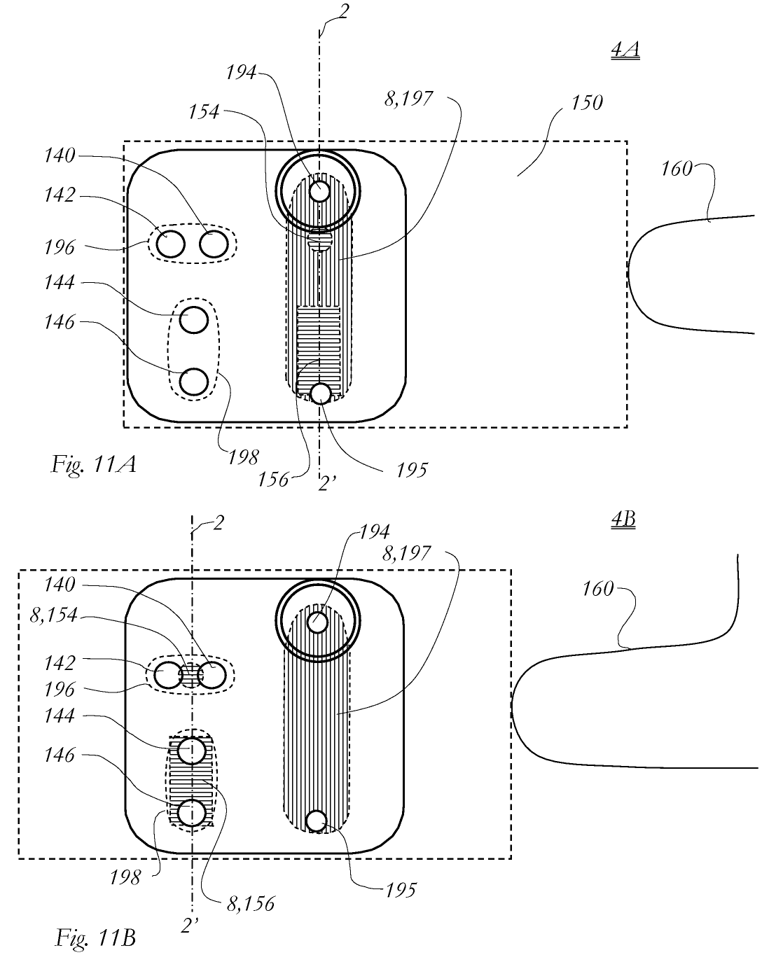

FIGS. 11A and 11B illustrates the sample volume isolation or segmentation mechanism. In FIG. 11A, sampling sled 150 is at its filling position 4A, and in FIG. 11B, sampling sled 150 is moved into its flushing position 4B, see the relative position of line 2-2′ of sampling sled 150. At the filling position 4A, filling inlet 194, venting aperture 195, and first and second sampling cavities 154 and 156 of sampling sled 150 are all aligned with line 2-2′ of sampling sled 150. As such, when blood 8 is filled in through filling inlet 194, blood 8 flows into first sampling cavity 154 and the second sampling cavity 156, and fills in recess 197 (see shaded area in FIG. 11A). The communication among filling inlet 194, recess 197, first and second sampling cavities 154 and 156, and venting aperture 195 can be further visualized in FIG. 10, which shows a cross-sectional view along line 2-2′ in FIG. 11A. During filling, the cassette is at its horizontal position, with filling inlet 194 in an upright position as shown in FIG. 10.

Subsequent to filling, sampling sled 150 is pushed into its flushing position 4B as shown in FIG. 11B, by pusher 160, or by an operator's hand. When first and second sampling cavities 154 and 156 of sampling sled 150 are moved away from recess 197, the blood above first and second cavities 154 and 156 is sheared off by edge 197a of recess 197 of sampling gasket 190 against the flat upper surface 152 of the sampling sled. As such, a predetermined volume of the blood is segmented or isolated in first sampling cavity 154 for red blood cell measurement and a predetermined volume of the blood is segmented or isolated in second sampling cavity 156 for white blood cell measurement, respectively. As shown in FIG. 11B, when sampling sled 150 is in flushing position 4B, the first cavity 154 is in communication with channels 140 and 142 which are in fluid communication with chambers 130 and 132, and the second cavity 156 is aligned with channels 144 and 146 which are in fluid communication with chambers 134 and 136.

In the process of measuring a blood sample, a disposable cassette 100 is placed into cassette compartment 30 of cassette receiving interface 20 at its open position, and a blood sample is filled through filling inlet 194 into sampling section 120 of the cassette. Then, cassette receiving interface 20 is moved promptly to the closed position. At this time, cassette interface 74 of blood measurement assembly 70 engages disposable cassette 100, with needles 74A, 74B, and 74C piercing into sample outlets 131 and 135 and cleaner outlet 139 (see FIG. 12). Needles 74A and 74B penetrate the divider within the sample outlets, which establishes fluid communications between the chambers and their respective channels. Then, the blood analyzer activates a pressure actuator assembly 90, which moves plunger 94 to apply a pressure on chamber 132, which causes the blood diluent to flow from chamber 132 through channel 142, through-hole 196, channel 140, into chamber 130. Pressure actuator assembly 90 also moves plunger 96 to apply a pressure on chamber 134, which causes the lytic reagent to flow from chamber 134 through channel 144, through-hole 198, channel 146, into chamber 136. As such, the channels, through-hole, and chambers within each pair are primed with the respective reagent contained therein.

At this time, the system control activates pusher 160, as the sampling activation mechanism of the blood analyzer, to push sampling sled 150 from filling position 4A to flushing position 4B. This movement of the sampling sled segments or isolates a first predetermined volume of the blood sample in the first cavity 154 and a second predetermined volume of the blood sample in the second cavity 156, respectively. Once sampling sled 150 is in the flushing position, pressure actuator assembly 90 moves plungers 94 and 96 forward to apply a pressure again on chambers 132 and 134. This time, the diluent in chamber 132 flows through channel 142, flushes the predetermined volume of blood 8 in first sampling cavity 154 into channel 140, and carries the blood into chamber 130, as illustrated in FIG. 11B. Similarly, the lysing reagent in chamber 134 flows through channel 144, flushes the predetermined volume of blood 8 in second sampling cavity 156 into channel 146, and carries the blood into chamber 136 (see FIG. 11B). Then, pressure actuator assembly 90 further applies a pressure alternatively between chambers 130 and 132 to mix the blood with the blood diluent, which forms the first sample mixture, and applies a pressure alternatively between chambers 134 and 136 to mix the blood with the lytic reagent, which forms the second sample mixture. It is noted that in FIG. 12, a phantom image of the cassette is shown for illustrating the engagement.

Subsequently, the first and second sample mixtures are drawn from sample outlets 131 and 135, respectively, through the needles and conduits connected thereto, into the two blood measurement devices for measuring red blood cell and white blood cell concentrations. After the measurements are complete, the cleaning solution in chamber 138 is drawn through outlet 139 into a conduit of the cassette interface, which is connected to the flow paths of the two blood measurement devices, to clean the flow paths and bring the sample mixtures back into chambers 130 and 132 and chambers 134 and 136. Then, cassette receiving interface 20 is moved to the open position, and the used cassette is disposed by the operator.

With the description of the disposable cassette and its use on the blood analyzer of the present invention, an electrical sensing mechanism operable with the electrical sensor type of the blood sensor is described now in reference to FIGS. 13A and 13B. As shown in FIG. 13A, disposable cassette 100 can further comprise a pair of electrodes 176a and 176b disposed within vent opening 175 of upper panel 112. The upper ends 76a and 76b of the electrodes are located on a side wall of housing 110 or on upper panel 112 forming an electrode interface, which is exposed for electrical contact, with surroundings sealed by diaphragm 116. The electrode interface is adapted to connect to an electrical sensor (not shown) in the cassette interface 20 of the blood analyzer, when the cassette is used on the blood analyzer. As illustrated in FIG. 13B, when blood is filled into sampling section through filling inlet 194, the blood flows into the first and second cavities 154 and 156, further fills in the space in vent opening 175, and typically with a small quantity entering space 172 above the vent opening. Therefore, during filling, electrodes 176a and 176b will immerse into the blood, which closes the circuitry. The electrical signal generated can be sensed by the electrical sensor of the blood analyzer, indicating the presence of blood in the cassette. The electrical sensor is connected to system control 80 and the time recording mechanism thereof, and the signal produced by the electrical sensor can be used for determining the blood dwelling time described hereinafter.

System control 80 of blood analyzer 10 comprises a time recording mechanism and a predetermined sedimentation time control criterion. The time recording mechanism records one or more selected period of time in the process of sample preparation for the purpose of controlling blood sedimentation. In one embodiment, the time recording mechanism is a digital or analog timer, which can be activated, or deactivated, by blood sensor, position sensor, and/or the sampling activation mechanism described above.

In one embodiment of the present invention, a first blood dwelling time and a second blood dwelling time can be recorded and used for controlling blood sedimentation during sample preparation. The first dwelling time is defined as a time period between a filling time at which the blood sensor detects the presence of a blood sample, as the sample is filled into sampling section 120 of cassette 100, and an engaging time at which cassette receiving interface 20 is moved to its closed position, or cassette compartment 230 of cassette receiving interface 220 is moved to its vertical position. The second dwelling time is defined as a time period between the engaging time and a sampling time at which a predetermined volume of the blood sample is isolated at sampling section 120 for measurement. When the movement of sample sled 150 is activated by the blood analyzer, the sampling time can be the time that the blood analyzer activates the sampling activation mechanism, because isolation or segmentation of a predetermined volume of the blood sample occurs instantly upon activation.

As can be appreciated from the sample preparation process described above, at the filling time cassette receiving interface 20 is at the open, substantially horizontal position, and disposable cassette 100 is also in a substantially horizontal position. After an operator introduces a blood sample through filling inlet 194, the blood fills in the entire space available within sampling section 120. FIGS. 14A and 14B illustrate enlarged partial cross sectional views of the sampling section of the cassette, along line A-A′ of the sampling sled in FIG. 8, with the cassette at horizontal and vertical positions, respectively, which show a blood sample 8 filled in the first sampling cavity 154 and in recess 197 (second sampling cavity not shown). As shown, at the horizontal position the blood cells in a volume of the blood above the first cavity 154 move downwardly, driven by gravity, and descend into the cavity when the cassette stays in this position. It has been found that if the cassette stays in the horizontal position for a period about 20 seconds, in other words, the first dwelling time exceeds such a time, sedimentation of the blood cells is sufficient to cause an increase of the red blood cell concentration reported by the blood analyzer. Further extension of the first dwelling time may cause an increase that exceeds the allowable error range required for clinical diagnosis purpose.

As can be appreciated, the same sedimentation phenomenon occurs to the blood in the second cavity 156, where the blood is used for measuring white blood cells. However, among the cells to be measured, i.e., red blood cells, platelets and white blood cells, the precision requirement for red blood cell concentration measurement in clinical diagnostic analysis is substantially higher than for other cell measurements, which typically has a required coefficient of variation (CV) of less than 1%. Typically, the required CV for platelet concentration measurement is less than 5%. Therefore, in terms of effect of sedimentation, red blood cell concentration (RBC) is the most sensitive parameter.

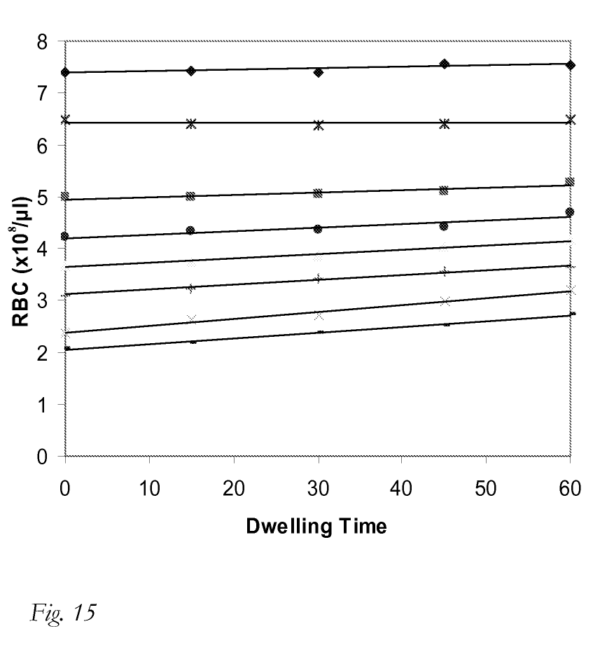

FIG. 15 illustrates the effect of sedimentation on the red blood cell concentration measured on a blood analyzer described above, which shows the reported red blood cell concentrations (RBC) of a blood sample with different first dwelling times during sample preparation process. It is noted that using disposable cassette 100 and blood analyzers of the present invention for measuring a blood sample, the average first dwelling time for operators of regular clinical laboratory skills is about 10 to 15 seconds, which has a minimum effect of sedimentation on the measurement of the red blood cell concentration, and the measurement results are well within required accuracy and precision ranges. However, to assess and illustrate potential effects of prolonged dwelling time, the results shown in FIG. 15 were obtained with first dwelling times extended substantially longer than normal process, which simulated poor operator performance or inadvertent situations. As shown in FIG. 15, the reported RBC increases substantially linearly with the first dwelling time. As can be further seen, the rate of increase of the reported RBC with the first dwelling time increases as the red blood cell concentration of a blood sample deceases. In other words, sedimentation appears having a stronger impact to the blood samples that have a relatively lower red blood cell concentration.

FIG. 14B illustrates the vertical orientation of sampling section 120 of disposable cassette 100 when cassette receiving interface 20 is moved to its closed position. In other words, during the second dwelling time, blood 8 is in this orientation. As can be appreciated, at this time the volume of the blood originally above the first cavity 154 and the second cavity 156 (not shown) is now on the side of the cavities. As such, sedimentation of the blood cells during the second dwelling time has a substantially less effect on the reported blood cell concentrations than that has during the first dwelling time. At the sampling time, the blood outside the first cavity 154 and the second cavity 156 is scraped away, and the blood within the cavities has no further contact with other portions of the blood sample in sampling section 120. Therefore, after isolation, during any further standing time before mixing the isolated blood with the reagent, no further sedimentation effect may impact the reported blood cell concentrations. In other words, the number of blood cells within the cavities remains constant, whether the blood cells suspend uniformly or decent toward the lower side of the cavities at the vertical orientation of the cassette. Hence, it can be understood that the concerned sedimentation effect is only present during the first and the second dwelling times, and is not present after isolation of the predetermined volume of the blood.

Further components of the system control and functionalities thereof are described hereinafter with regard to controlling sample preparation process and measurements on the blood analyzer to prevent sedimentation of blood cells to affect the accuracy of the measurements of blood samples.

System control 80 can be a microprocessor with a system control program. In one embodiment, the system control program comprises a predetermined sedimentation time control criterion that includes an upper limit of the first dwelling time. The predetermined sedimentation time control criterion can further include other suitable parameters, for example, an upper limit of the second dwelling time, as further described later. The system control further comprises a sedimentation evaluation mechanism operable to evaluate a recorded dwelling time of a blood sample in reference to the predetermined sedimentation control criterion, and predetermined sample analysis instructions, such as proceed-further instruction, flagging instruction, and abortion instruction, one or more is generated by the sedimentation evaluation mechanism based on the result of evaluation, as described in further detail below.

In a sample analysis process described above, when the operator introduces a blood sample into sampling section 120 of cassette 10 through filling inlet 194, the time recording mechanism is activated by blood sensor 40 to record the first dwelling time. Then, the sedimentation evaluation mechanism of system control 80 compares the recorded first dwelling time with an upper limit preset in the sedimentation time control criterion, and generates a sample analysis instruction, based on the result of the comparison or evaluation. When the recorded first dwelling time does not exceed the upper limit, a proceed-further instruction is issued by the sedimentation evaluation mechanism. With this instruction, blood measurement assembly 70 proceeds engaging with the disposable cassette to deliver the first and second sample mixtures into the flow paths for measuring the red blood cell and white blood cell concentrations, as well as hemoglobin concentration. The results of the measurements are reported on a blood analysis report.

When the recorded first dwelling time exceeds the upper limit, a flagging instruction may be generated by the sedimentation evaluation mechanism. Under such an instruction, blood measurement assembly 70 proceeds with the measurement as described above; however, a sedimentation warning is generated on the blood analysis report, or alternatively, only the sedimentation warning is generated without results of the measurements.

Moreover, when the recorded first dwelling time exceeds the upper limit, instead of issuing a flagging instruction, an abortion instruction may be issued by the sedimentation evaluation mechanism. Under such an instruction, the subsequent steps of sample preparation, such as isolation of a predetermined volume of the blood and mixing the blood with the reagents, as well as measurement of the blood sample mixture by blood measurement assembly 70 are completely aborted. In this situation, an error message can also be provided through user interface 88 to request the operator rerun this blood sample with a new cassette.

The sedimentation evaluation mechanism of the system control can be a computer program including an algorithm designed to perform comparison of the recorded dwelling time with the defined upper limit thereof, and/or evaluation of other parameters of the predetermined sedimentation control criterion, and to generate the sample analysis instructions, or decisions.

As mentioned above, the predetermined sedimentation time control criterion can further comprise an upper limit of the second dwelling time. In this situation, the sedimentation evaluation mechanism of the system control compares the recorded first dwelling time and the recorded second dwelling time with their respective upper limits preset in the sedimentation time control criterion, and generates a sample analysis instruction based on the result of the comparison, as described above.

In the embodiment described above, sample sled 150 is moved from the filling position 4A to the flushing position 4B by pusher 160 which is activated by system control 80. For an automated operation, system control 80 further comprises a sampling instruction, which activates the sampling activation mechanism when position sensor 60 detects that cassette receiving interface 20 has moved into the closed position. As can be appreciated, with the automated operation, the second dwelling time can be substantially a constant for all blood samples prepared on the blood analyzer, unless instrument malfunction occurs. Therefore, the upper limit of the second dwelling time can also function as an instrument reliability criterion.

In an alternative embodiment, sample sled 150 can be moved from the filling position 4A to the flushing position 4B manually by the operator, when disposable cassette 100 is in the horizontal position and cassette receiving interface 20 is at its open position. In this embodiment, side wall 32A of the cassette compartment can have an opening for access by the operator. In this situation, isolation of predetermined volumes of the blood occurs when the first cavity 154 and the second cavity 156 are at the horizontal position shown in FIGS. 13B and 14. After isolation, the blood above the two cavities is scraped away; therefore, if the cassette remains in the horizontal position for an additional time, or remains idle for an additional time after cassette receiving interface 20 is moved to the closed position, no further effect of sedimentation affects the blood cell concentration measurement. Under such a circumstance, the first dwelling time described above becomes the only dwelling time during which sedimentation needs to be considered.

For automated operation, system control 80 can further comprise a starting criterion, which includes a ready indication for starting analysis of a blood sample, in other words, starting a new cycle of sample preparation and measurement after completion of measurements of a prior blood sample. In one embodiment, when position sensor 60 detects an absence of cassette receiving interface 20 at the closed position and cassette sensor 50 detects an absence of disposable cassette 100 in cassette compartment 30, which means the used cassette has been removed and cassette receiving interface 20 is in open position for receiving a new cassette, system control 80 may issue a ready indication for resetting the timer and the data processor for starting analysis of a new sample. As can be understood, at this time blood sensor 40 should also detect an absence of blood.

Moreover, to prevent an operator filling a blood sample on the bench then loading the filled cassette onto the blood analyzer, the starting criterion of system control 80 can include a prerequisite on the order and/or the time interval between loading a cassette into cassette compartment 30 and filling a blood sample. Such a prerequisite requires that the cassette sensor detects loading of a cassette into cassette compartment 30 before the blood sensor detects filling of a blood sample into the cassette. Otherwise, system control 80 prohibits starting analysis of a blood sample. If an operator fills a blood sample into the cassette on a bench and then moves the already filled cassette into cassette compartment 30, the cassette sensor and the blood sensor will detect the presence of the cassette and the blood as the same time. This fails to meet the prerequisite, and system control 80 will not start sample preparation process described above.

In addition, the blood analyzer of the present invention can further include one or more user restriction mechanisms. In one embodiment, the blood analyzer further includes an alarm connected to the time recording mechanism or system control 80. When a blood sample is filled into the cassette, once the recorded time exceeds a predetermined warning limit, for example 10 seconds, the time recording mechanism or system control 80 triggers the alarm, which reminds the operator to move the cassette receiving interface to the closed position. Alternatively, the cassette receiving interface can be moved to the closed position automatically once the recorded time exceeds a predetermined warning limit. In a further embodiment, the blood analyzer can include a spring loaded door panel instead of a hinged door panel as shown in FIG. 2. With a spring loaded door panel, the operator needs to hold the door panel open with one hand for filling a blood sample, and the door will be closed once the operator release the hand. This structure can naturally reduce the incidences of extended first dwelling time due to operator's delay.

In addition to the sedimentation control mechanism discussed above, optionally cassette receiving interface 20 can further include a motion activator, such as a vibrator disposed within the door panel, to cause movement of blood cells in the blood filled within sampling section 120 to retard sedimentation.

As can be appreciated, the blood analyzer of the present invention having the sedimentation control mechanism described above can monitor and control the blood dwelling time and to prevent sedimentation from affecting accuracy of the reported parameters. It should be understood, although the present invention has been described particularly in reference to hematology analyzers which are directly related to cellular particle counting, the sedimentation control mechanism of the present invention can be used for other blood analysis instruments where sedimentation of the cellular particles in a blood sample is a concern. Moreover, the sedimentation control mechanism of the present invention can also be used for monitoring and controlling particle sedimentation of other particle suspensions, biological or non-biological particle suspensions in sample isolation and measurement processes.

While the present invention has been described in detail and pictorially shown in the accompanying drawings, these should not be construed as limitations on the scope of the present invention, but rather as an exemplification of preferred embodiments thereof. It will be apparent, however, that various modifications and changes can be made within the spirit and the scope of this invention as described in the above specification and defined in the appended claims and their legal equivalents.

1. A blood analyzer comprising:
(a) a cassette receiving interface comprising a cassette compartment and a blood sensor operable to detect a presence of blood in a disposable cassette removably disposed within said cassette compartment;
(b) a system control connected to said blood sensor, said system control comprising a time recording mechanism and a predetermined sedimentation time control criterion; and
(c) a blood measurement assembly connected to said system control, and adapted to connect with said disposable cassette.

2. The blood analyzer of claim 1, wherein said blood sensor comprises an optical sensor or electrical sensor.

3. The blood analyzer of claim 1, wherein said sedimentation time control criterion comprises an upper limit of a dwelling time, said dwelling time being defined as a time period between a filling time at which said blood sensor detects a blood sample being filled into said cassette and a sampling time at which a predetermined volume of said blood sample is isolated in said cassette for measurement.

4. The blood analyzer of claim 3, wherein said system control further comprises a sedimentation evaluation mechanism operable to evaluate a recorded dwelling time of said blood sample in reference to said predetermined sedimentation control criterion.

5. The blood analyzer of claim 1, wherein said cassette receiving interface is movable between a first position and a second position and said blood analyzer further comprises a position sensor electrically connected to said system control, operable to detect a position of said cassette receiving interface.

6. The blood analyzer of claim 5, wherein said position sensor comprises a mechanical, electrical, or optical sensor.

7. The blood analyzer of claim 5, further comprising a sampling activation mechanism adapted to engage with said cassette to initiate isolation of a predetermined volume of a blood sample filled into said cassette.

8. The blood analyzer of claim 7, wherein said sedimentation time control criterion comprises an upper limit of a first dwelling time, said first dwelling time is defined as a time period between a filling time at which said blood sensor detects a blood sample being filled into said cassette and an engaging time at which said cassette receiving interface is moved to said second position.

9. The blood analyzer of claim 8, wherein said system control further comprises a sedimentation evaluation mechanism operable to evaluate a recorded dwelling time of said blood sample in reference to said predetermined sedimentation control criterion.

10. The blood analyzer of claim 9, wherein said system control further comprises predetermined sample analysis instructions, including a proceed-further instruction, a flagging instruction, or an abortion instruction.

11. The blood analyzer of claim 8, wherein said system control further comprises a sampling instruction, said sampling instruction initiating said sampling activation mechanism to isolate said predetermined volume of said blood sample in said cassette for measurement, when said position sensor detects said cassette receiving interface moving into said second position.

12. The blood analyzer of claim 11, wherein said sedimentation time control criterion further comprises an upper limit of a second dwelling time, said second dwelling time being defined as a time period between said engaging time and a sampling time at which a predetermined volume of said blood sample is isolated in said cassette for measurement.

13. The blood analyzer of claim 12, wherein said system control further comprises a sedimentation evaluation mechanism operable to evaluate a recorded dwelling time of said blood sample in reference to said predetermined sedimentation control criterion.

14. The blood analyzer of claim 13, wherein said system control further comprises predetermined sample analysis instructions, including a proceed-further instruction, a flagging instruction, or an abortion instruction.

15. The blood analyzer of claim 11, wherein said cassette receiving interface further comprises a cassette sensor, electrically connected to said system control, operable to detect a presence of said disposable cassette in said cassette compartment.

16. The blood analyzer of claim 15, wherein said system control further comprises a starting criterion, said starting criterion comprises a ready indication for starting sample analysis when said position sensor detects an absence of said cassette receiving interface at said second position and said cassette sensor detects an absence of said disposable cassette in said cassette compartment.

17. The blood analyzer of claim 15, wherein said system control further comprises a prerequisite on an order of, or a time interval between, placing said cassette into said cassette compartment and filling of said blood sample into said cassette.

18. The blood analyzer of claim 1, wherein said cassette receiving interface further comprises a motion activator, adapted to affect said disposable cassette disposed within said cassette compartment to cause movement of particles in said blood in said cassette to retard sedimentation.

19. A method of controlling blood cell sedimentation during sample preparation on a blood analyzer, said method comprising:
(a) providing a blood analyzer comprising a cassette receiving interface that includes a cassette compartment and a blood sensor, a blood measurement assembly, and a system control electrically connected to said blood sensor and said blood measurement assembly, said system control comprising a time recording mechanism and a predetermined sedimentation time control criterion;
(b) placing a disposable cassette into said cassette compartment, and filling a blood sample into said disposable cassette;
(c) isolating a predetermined volume of said blood sample in said cassette;
(d) recording a dwelling time using said time recording mechanism, said dwelling time being defined as a time period between a filling time at which said blood sensor detects said blood sample being filled into said cassette and a sampling time at which said predetermined volume of said blood sample is isolated in said cassette;
(e) comparing recorded dwelling time of said blood sample with an upper limit of said dwelling time in said predetermined sedimentation time control criterion; and
(f) generating a sample analysis decision based on a result obtained in (e).

20. The method of claim 19, further comprising measuring said blood sample in said blood measurement assembly, when said sample analysis decision is a proceed-further instruction generated when recorded dwelling time does not exceed said upper limit.

21. The method of claim 19, further comprising measuring said blood sample in said blood measurement assembly and generating a sedimentation warning on a blood analysis report, when said sample analysis decision is a flagging instruction generated when recorded dwelling time exceeds said upper limit.

22. The method of claim 19, further comprising aborting measuring said blood sample in said blood measurement assembly, when said sample analysis decision is an abortion instruction generated when recorded dwelling time exceeds said upper limit.
